(19) United States
(12) Patent Application Publication
(10) Pub. No.: US 2024/0307612 A1
(43) Pub. Date: Sep. 19, 2024

(54) INFUSION DEVICE, INFUSION DEVICE COMPONENTS, AND METHOD OF DETERMINING FLOW RATE

(71) Applicant: Amgis, LLC, Medina, NY (US)

(72) Inventors: Jason Maine, Hamlin, NY (US); James McGarvey, Hamlin, NY (US); Kevin Cifelli, Medina, NY (US); Roger Hungerford, Kissimmee, FL (US)

(21) Appl. No.: 18/606,929

(22) Filed: Mar. 15, 2024

Related U.S. Application Data
(60) Provisional application No. 63/490,265, filed on Mar. 15, 2023.

(51) Int. Cl. A61M 5/142; G06T 1/00

(52) U.S. Cl. CPC A61M 5/1424; G06T 1/0007; A61M 2205/10; A61M 2205/3334

(57) ABSTRACT

An infusion device. individual components of the infusion device, and a method related to obtaining fluid measurements provided by the infusion device. A specialty fluid chamber arranged to afford optimal optical imaging clarity by a camera of fluid therein or entering the chamber.

### CROSS-REFERENCE TO RELATED APPLICATIONS

This application claims priority pursuant to 35 U.S.C. 119(e) to U.S. Provisional Patent Application No. 63/490,265, titled, Infusion Device, Infusion Device Components, and Method of Determining Flow Rate, filed Mar. 15, 2023, which application is incorporated herein by reference in its entirety.

### FIELD

The invention generally relates to an infusion device, individual components of the infusion device, and methods related to obtaining fluid measurements provided by the infusion device.

### BACKGROUND

Infusion devices are well known in the art and encompass a plurality of variations, designs, and methods of administering and controlling a selected fluid flow to a patient, such as gravity infusion sets or pump-driven sets. In some designs, a single camera system is utilized to capture a liquid drop passing through a drip chamber wherein a captured image of the drop is processed to determine individual drop size, drop growth, and drop volume. The combination of these measurements affords an on-board computer to process and calculate a flow rate.

As stated above, infusion devices traditionally take one of two forms, a gravity infusion set, or a pump-driven infusion set. The gravity infusion set allows the flow of the fluid through the action of the gravitational force. Therefore, the source of fluid is always higher than the location of the patient receiving the infusion. The higher the height of the infusion bottle, the stronger the force of gravity that will act on the flow of fluid. Inversely, the pump-driven infusion set pushes the flow of fluid using the force coming from the pump. Gravity infusion devices are preferred as pump-driven sets can complicate the infusion process by delivering a selected fluid at too high of a flow rate—causing medical issues.

Infusion devices traditionally comprise at least one fluid container, e.g., IV bags, arranged to house a selected infusion fluid, e.g., water, medications or electrolytes, nutrition, and/or blood, a drip chamber which serves as the venue for determining a fluid flow rate and is in fluid connection with the at least one fluid container, a flow regulator (or flow control valve) which is arranged downstream (i.e., distally) in relation to the drip chamber and arranged to control the flow of fluid from the drip chamber to an injection site within a patient. All of the aforementioned components are in fluid communication vis-à-vis tubing which is traditionally flexible.

Currently, drip chambers are constructed with a flexible material and are flexible. These drip chambers also have curvature in their wall design. Although this material choice provides benefits such as allowing air to be physically depressurized, e.g., squeezing, from the drip chamber, the flexible material can easily become distorted, making visual captures of the drip chamber (via a camera) impossible to provide adequate images for fluid measurement processing and/or calculation. Even without the flexible drip chambers being subject to distortion, the curvature can create image distortion as well.

Traditionally, the images captured by the camera are used to determine flow rate, fluid volume, etc.—however, there is not a method for measuring flow rate directly. This results in inaccurate delivery of the selected fluid, unreliably detection of flow disruptions (e.g., under-detection, over-detection, and/or delayed-detection), and an inability to detect deadly catastrophic hardware failure and/or failures.

During the course of an infusion delivery, a fluid container can run dry, requiring the IV line (e.g., tubing) to be disconnected from a patient to remove air from the line. Disconnecting the patient necessarily increases the total duration of a fluid delivery, requires additional assistance from a medical professional, and increases the risk of potential infection during the disconnection and reconnection.

Therefore, there is a long felt need for:
1. A device arranged for a gravity infusion set that allows pressure to be introduced to the fluid line, thereby allowing flow rate of the fluid to be increased;
2. A drip chamber for an infusion set that is not flexible, is flat-wall and is constructed of a material allowing images of fluid and/or fluid drops captured within the drip chamber to be of the highest resolution and/or quality (i.e., not distorted);
3. A device arranged for an infusion set that allows excess air to be released from the drip chamber without disconnecting and reconnecting a patient;
4. A method for directly calculating the volume of fluid entering a drip chamber of an infusion system; and,
5. A method for directly calculating the flow rate of fluid being infused by an infusion system.

### SUMMARY

The present invention is an infusion device having a variety of sub-devices connected therein, specifically devices arranged to release air pressure from a fluid chamber and a device allowing manual stimulation of a pump to increase flow rate. Additionally, the present invention also comprises a method of directly measuring volume of a fluid entering a fluid chamber, and fluid flow rate. Further, the present invention also comprises a fluid chamber, or drip chamber, having a transparent and rigid construction in combination with “flat-walls” to allows for optimal optical imaging clarity of fluid and/or a fluid drop, therein.

A general object of the invention is to provide a device arranged for a gravity infusion set that allows pressure to be introduced to the fluid line, thereby allowing flow rate of the fluid to be increased.

Another object is to provide a drip chamber for an infusion set that is not flexible, is flat-wall and is constructed of a material allowing images of fluid and/or fluid drops captured within the drip chamber to be of the highest resolution and/or quality (i.e., not distorted).

A further object is to provide a device arranged for an infusion set that allows excess air to be released from the drip chamber without disconnecting and reconnecting a patient.

Still another object is to provide a method for directly calculating the volume of fluid entering a drip chamber of an infusion system.

Still a further object is to provide a method for directly calculating the flow rate of fluid being infused by an infusion system.

These and other objects, features, and advantages of the present invention will become readily apparent upon a review of the following detailed description of the invention, in view of the drawings and appended claims.

### BRIEF DESCRIPTION OF THE DRAWINGS

Various embodiments are disclosed, by way of example only, with reference to the accompanying schematic drawings, in which corresponding reference symbols indicate corresponding parts, in which:

FIG. 1 illustrates a schematic of an embodiment of an infusion device of the present invention;

FIG. 2 illustrates a perspective view of the exterior of the infusion device;

FIG. 3A illustrates a perspective view of the exterior of a fluid chamber;

FIG. 3B illustrates the perspective view shown in FIG. 3A with a portion of the exterior of the fluid chamber cutaway;

FIG. 4A-4H illustrates a representative cross-sectional of the fluid chamber in FIG. 3A and generally illustrates representative fluid flow patterns therein;

FIG. 5 illustrates a representative camera assembly operationally coupled with the fluid chamber; and,

FIG. 6 illustrates a representative method of determining fluid volume flow through an infusion device.

### TERMINOLOGY

The following terms and/or phrases used within this specification should be interpreted as follows:

The term “downstream”, and/or equivalents thereof, is intended to mean the direction that fluid flows. Equivalents, such as “distal”, e.g., “a distal outlet”, should be interpreted similarly with respect to fluid interactions with a specific component and considers a spatial relationship.

The term “upstream”, and/or equivalents thereof, is intended to mean the opposite direction from the direction that the fluid flows. Equivalents, such as “proximal”, e.g., “component X is located proximally in relation to component Y”, e.g., should be interpreted similarly with respect to fluid interactions with a specific component and should be considered as a spatial relationship.

The aforementioned terms, “downstream” and “upstream”, can be used interchangeably to describe characteristics, arrangements, and interactions of components, but should still be interpreted according to the definitions set forth, supra. For example, “component X is arranged upstream in relation to component Y” can mean that fluid flows from “component X” to “component Y”, inversely, it can also be said that “component Y is located downstream in relation to component X”, meaning that fluid flows from “component X” to “component Y”.

It should be noted that the terms “upstream” and “downstream”, used herein, do not contradict known understandings of pressure interactions of fluid and/or gases. For example, if a component is pressurized with a gas and subsequently depressurized upstream of the component, the gas will subsequently flow upstream.

The aforementioned terms, “distal” and “proximal”, should be understood in view of the definitions of “downstream” and “upstream” but also imply a spatial relationship between components, e.g., “component X includes proximal port and distal port”, where the “proximal port” is arranged above the “distal port”—fluid flows from the “proximal port” to the “distal port”.

The phrases “fluid connection”, “fluidly connected”, “in fluid communication”, “fluid communication”, and equivalents thereof, is intended to mean that two or more components are connected such that a substance, e.g., gases, fluid, etc., can flow between and/or within the two components.

The phrases “electrical communication”, “electrical connection”, “electrically connected”, “in electrical connection”, and/or equivalents thereof, mean either data communication between two components, electrical current between two components, or a combination thereof between two components.

### DETAILED DESCRIPTION

At the outset, it should be appreciated that like drawing numbers on different drawing views identify identical, or functionally similar, structural elements. It is to be understood that the claims are not limited to the disclosed aspects.

Furthermore, it is understood that this disclosure is not limited to the particular methodology, materials and modifications described and, as such, may, of course, vary. It is also understood that the terminology used herein is for the purpose of describing particular aspects only and is not intended to limit the scope of the claims.

Unless defined otherwise, all technical and scientific terms used herein have the same meaning as commonly understood to one of ordinary skill in the art to which this disclosure pertains. It should be understood that any methods, devices or materials similar or equivalent to those described herein can be used in the practice or testing of the example embodiments.

It should be appreciated that the term “substantially” is synonymous with terms such as “nearly,” “very nearly,” “about,” “approximately,” “around,” “bordering on,” “close to,” “essentially,” “in the neighborhood of,” “in the vicinity of,” etc., and such terms may be used interchangeably as appearing in the specification and claims. It should be appreciated that the term “proximate” is synonymous with terms such as “nearby,” “close,” “adjacent,” “neighboring,” “immediate,” “adjoining,” etc., and such terms may be used interchangeably as appearing in the specification and claims. The term “approximately” is intended to mean values within ten percent of the specified value.

It should be understood that use of “or” in the present application is with respect to a “non-exclusive” arrangement, unless stated otherwise. For example, when saying that “item x is A or B,” it is understood that this can mean one of the following: (1) item x is only one or the other of A and B; (2) item x is both A and B. Alternately stated, the word “or” is not used to define an “exclusive or” arrangement. For example, an “exclusive or” arrangement for the statement “item x is A or B” would require that x can be only one of A and B. Furthermore, as used herein, “and/or” is intended to mean a grammatical conjunction used to indicate that one or more of the elements or conditions recited may be included or occur. For example, a device comprising a first element, a second element and/or a third element, is intended to be construed as any one of the following structural arrangements: a device comprising a first element; a device comprising a second clement; a device comprising a third element; a device comprising a first element and a second element; a device comprising a first element and a third element; a device comprising a first element, a second element and a third element; or, a device comprising a second element and a third element.

Moreover, as used herein, the phrases “comprises at least one of” and “comprising at least one of” in combination with a system or element is intended to mean that the system or element includes one or more of the elements listed after the phrase. For example, a device comprising at least one of: a first element; a second element; and, a third element, is intended to be construed as any one of the following structural arrangements: a device comprising a first clement; a device comprising a second element; a device comprising a third element; a device comprising a first element and a second element; a device comprising a first element and a third element; a device comprising a first element, a second element and a third element; or, a device comprising a second element and a third clement. A similar interpretation is intended when the phrase “used in at least one of:” is used herein.

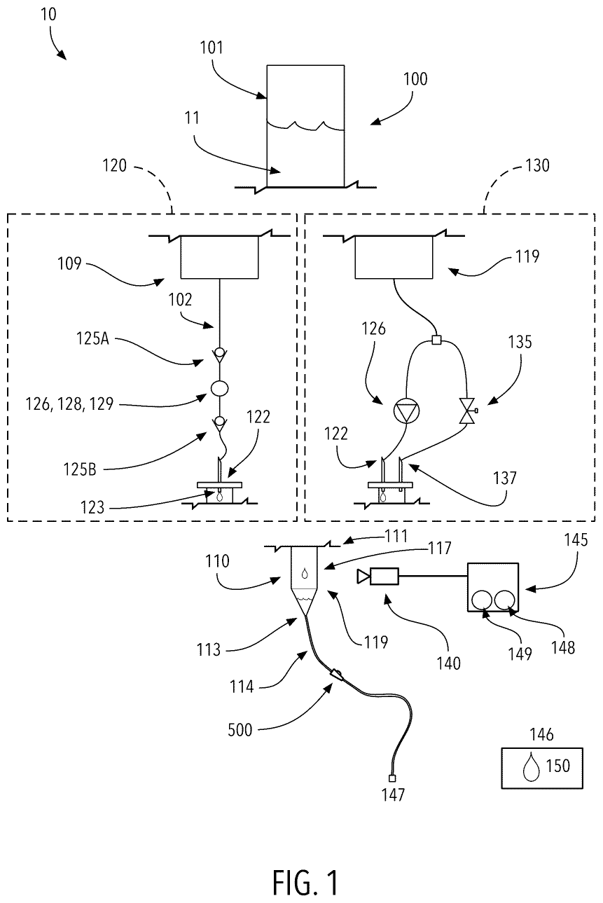

Adverting to the drawings, FIG. 1 depicts an infusion device 10 having either infusion delivery device 120, an air pressure release device 130, or both.

FIG. 1 illustrates, generally, and in the representative embodiments, the infusion device 10 of the present invention that includes a fluid container 100, e.g., an IV bag, or other container used in medical settings to hold an infusion fluid 11, a fluid chamber 110 in fluid communication with the fluid container 100 and arranged downstream of the fluid container 100, and an outlet line arranged downstream of the fluid chamber 110. The outlet line is preferably arranged to be in fluid communication with a patient, e.g., at a venipuncture cite 147, or similar patient to IV line connection. Infusion device 10 also includes at least one camera 140 that is in electrical communication with at least one processor 145, i.e., the camera 140 could be in electrical communication with a primary processor 145A and a secondary processor 145B, etc.

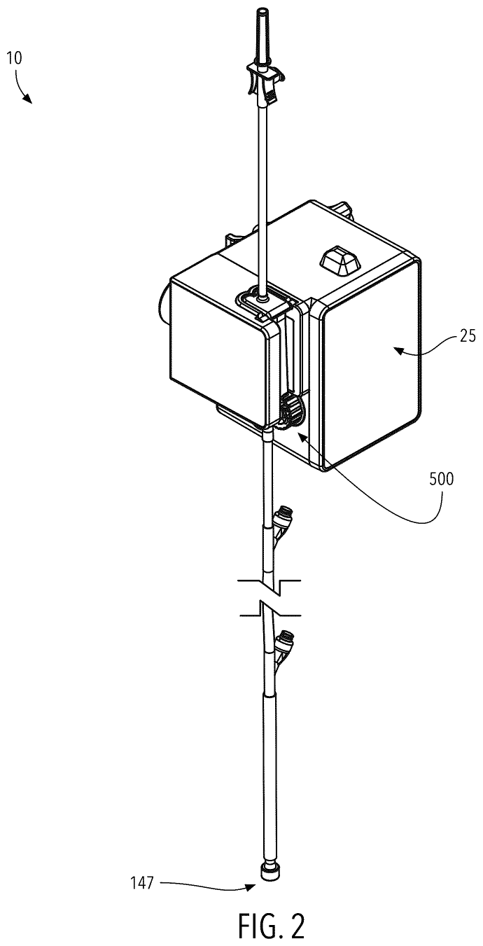

The camera 140 is arranged to have a field of view such that the fluid chamber 110 can be fully viewed, i.e., single image capture and/or video capture and appears within, as illustrated in FIG. 2, an external container portion 25 of the infusion device 10. The images and/or videos 146 are arranged to be communicated to the processor 145, where the processor 145 is arranged to be programmed with calculating software 148 that uses the images and/or videos 146 to make plurality of different fluid measurements relating to the fluid chamber 110.

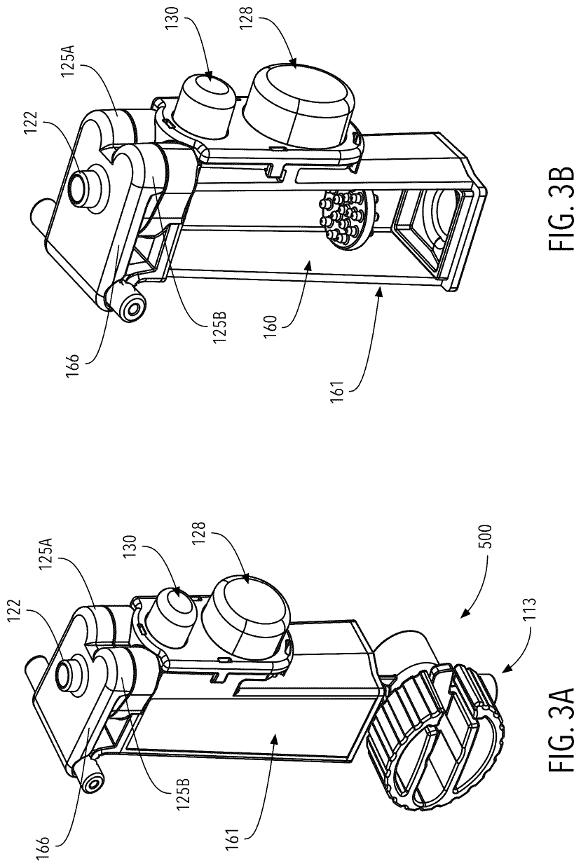

The fluid chamber 110 in some embodiments is a drip chamber having at least an inlet port 122 for fluid 11, a drop former 123 in fluid communication with the inlet port 122—and as introduced in FIG. 3A-3B—an internal cavity 160 defined by a body 161 and in fluid communication with the drop former 123, and a fluid outlet port 113 in fluid communication with the internal cavity 160. The drop former 123 is arranged to allow fluid 11 coming from the fluid container 100 by way of the fluid inlet port 122 with a drip chamber cap 166 to enter the fluid chamber 110 in single drops 150.

In some embodiments, the infusion device 10 could have an air pressure release device 130 in fluid communication with the fluid container 100 and the fluid chamber 110, arranged therebetween, i.e., downstream from the fluid container 100 and upstream from the fluid chamber 110. In other embodiments the infusion device 10 could have an infusion delivery device 120 in fluid communication with the fluid container 100 and the fluid chamber 110, arranged therebetween, i.e., downstream from the fluid container 100 and upstream from the fluid chamber 110. In further embodiments, the infusion device 10 could include both the air pressure release device 130 and the infusion delivery device 120, i.e., the infusion delivery device 120 could be located where the pump 126 is generally depicted in the air pressure release device 130.

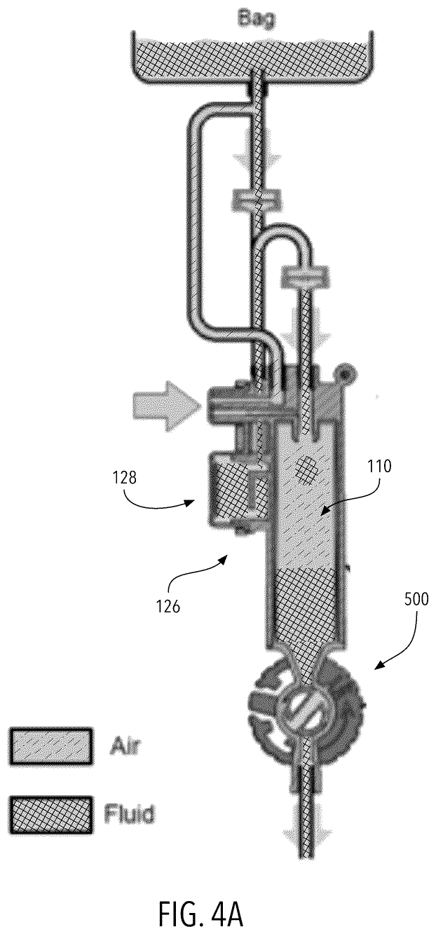

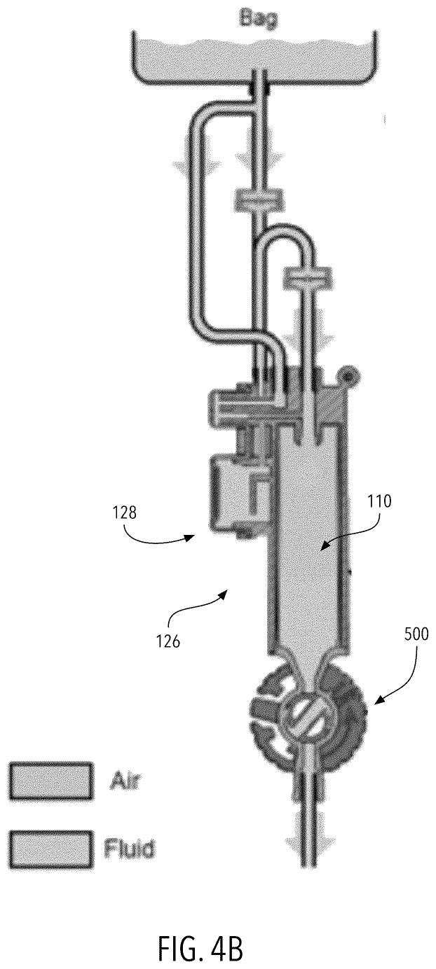

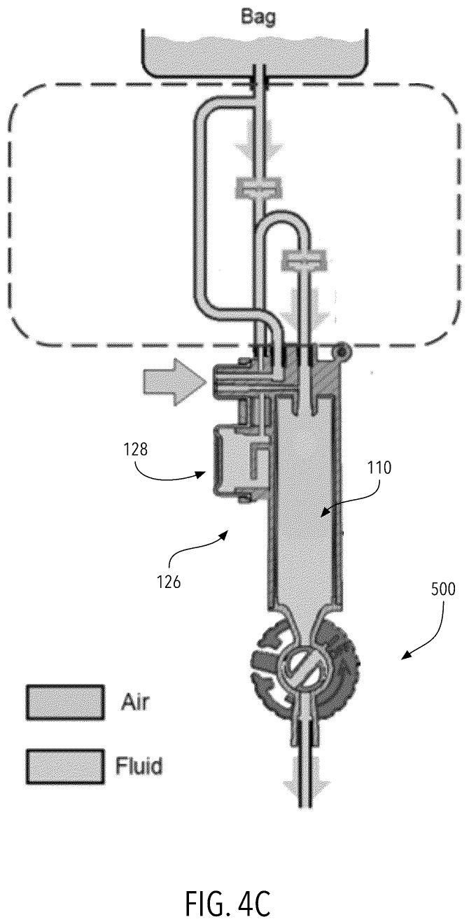

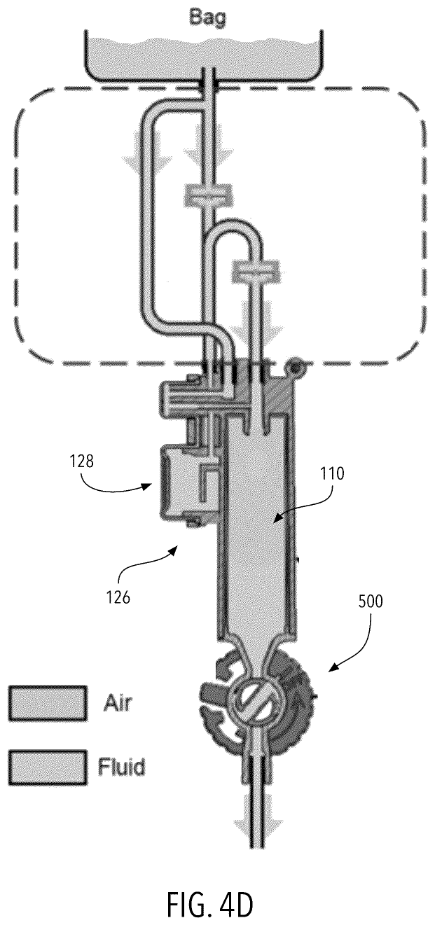

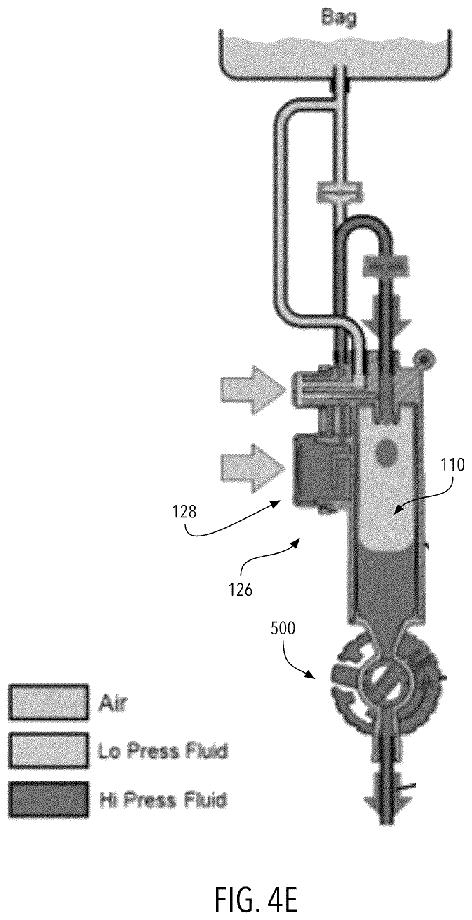

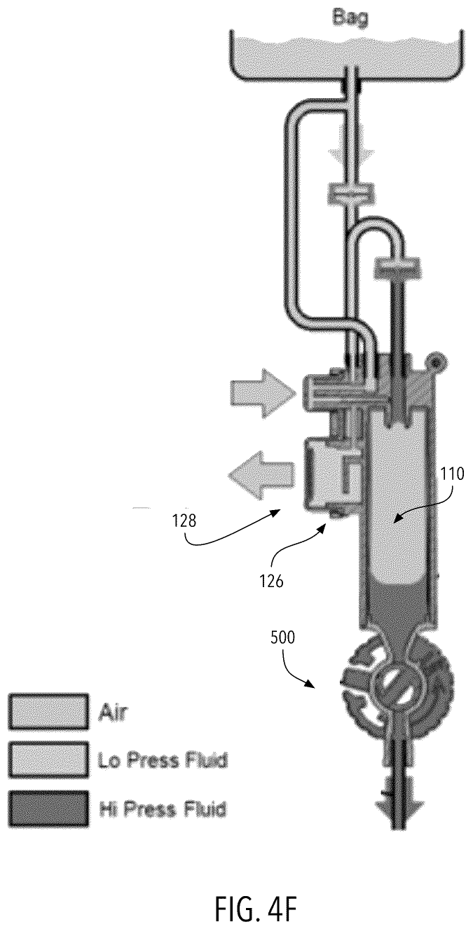

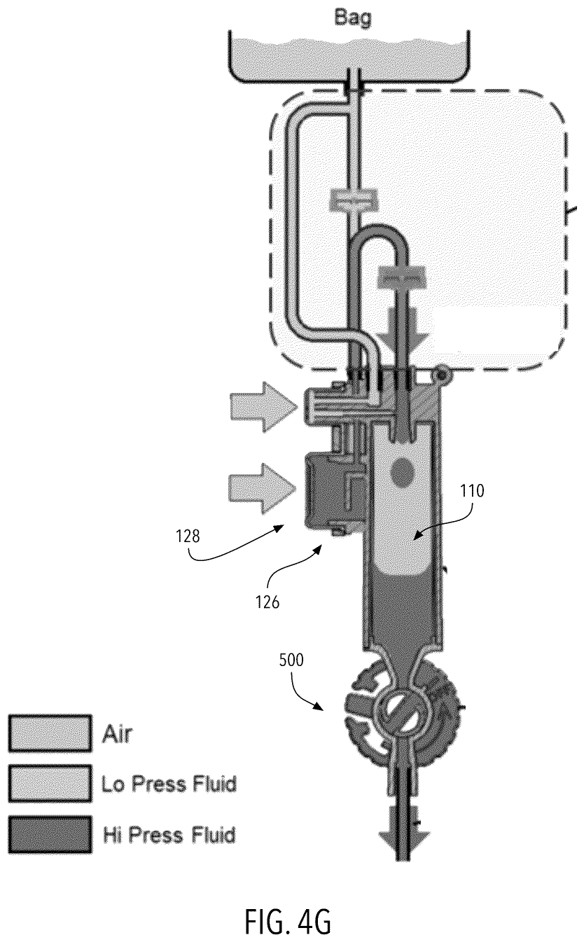

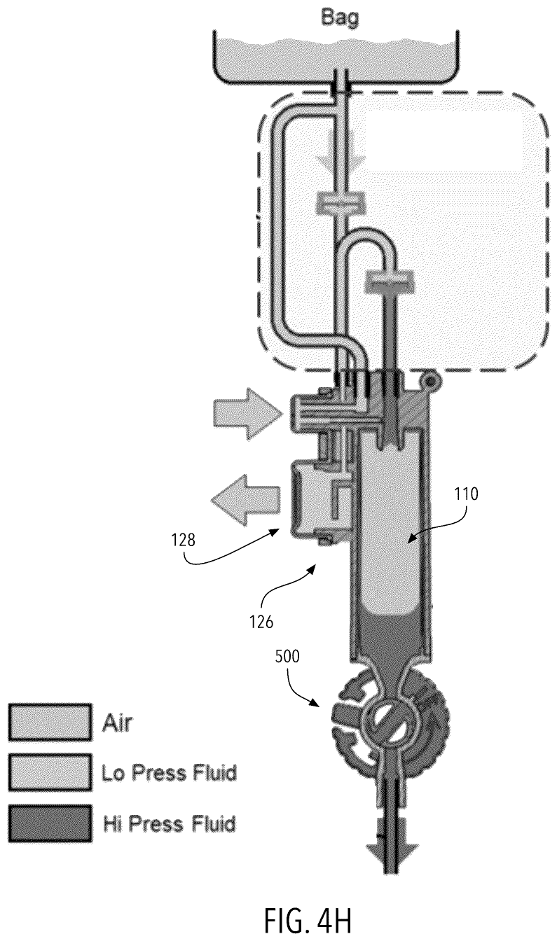

The air pressure release device 130 is arranged to allow the downstream line to fluidly connect the fluid container 100 and the fluid chamber 110 to be filled along with the fluid chamber 110 via the pump 126, however, it should be appreciated that the pump 126 is not a required component as the device is arranged to allow fluid flow in a downstream direction via gravity. FIGS. 4A-4H, however, illustrate a representative embodiment of the fluid chamber 110 and methods of operation including the pump 126.

Generally, and in the representative embodiments, the air pressure release device 130 comprises an infusion device 10 having a fluid container 100 having a proximal end 101 and a distal end 109, a fluid pump 126 arranged downstream of the fluid container 100 and in fluid communication with the fluid container 100, a fluid chamber 110 having a proximal end 111 and a distal end 119, the fluid chamber 110 arranged downstream of the fluid pump 126 and in fluid communication with the fluid pump 126, the fluid chamber 110 including a fluid inlet port 122 arranged at the proximal end of the fluid chamber 111 and in fluid communication with the fluid pump 126, the fluid inlet port 122 having a first check valve arranged upstream 125A, an air outlet port 137 arranged on a proximal end of the fluid chamber 111 and in fluid communication with the distal end of the fluid container 109, the air outlet port 137 having an air control valve 135 arranged downstream in relation to the fluid container 100 and further arranged upstream in relation to the fluid chamber 110, and a fluid outlet port 113 arranged on a distal end of the fluid chamber 119 and further including a fluid control valve 500, the fluid outlet port 113 is in fluid communication with a fluid outlet line 114 arranged downstream the fluid control valve 500, and a camera 140 in electrical communication with at least one processor 145 and having a field of view of the fluid chamber 110.

Generally, and in the representative embodiments, the aforementioned infusion device 10 and air pressure release device 130 automatically fills the fluid outlet line 114 leading up to a fluid chamber 110, and the fluid chamber 110 itself, by pumping fluid into the drip chamber, then releasing the resulting air pressure by opening a valve, where the released air then travels upstream and into the fluid container 100. This generally functions by closing the fluid outlet port 113 of the fluid chamber 110 and closing the air outlet port 137 of the fluid chamber 110 and then using the pump 126 to move fluid 11 from the fluid container 100 into the fluid chamber 110 via the pump 126 through the fluid inlet port 122 of the fluid chamber 110 until fluid 11 in the fluid chamber 110 reaches a preferable volume. Once the preferable volume of the fluid 11 in the chamber is achieved the inlet port 122 of the fluid chamber 110 is closed—resulting in an increase of air pressure within the fluid chamber 110. Subsequently, the air control valve 135 is opened thereby releasing the pressurized air upstream (via the pressure) into the fluid container 100, or in alternative embodiments, the pressurized air is released into a secondary container in fluid communication with the air control valve 135, or in further embodiments, the pressurized air is released to the open external environment.

Therefore, the fluid inlet port 122 of the fluid chamber 110 includes a fluid control valve 500, which could comprise a check valve (one-way) 125 or a controllable valve (mechanical, electronic, etc.). The air outlet port 137 of the fluid chamber 110 includes an air control valve 135 which is preferably a controllable valve (mechanic, electronic, etc.) The fluid outlet port 113 of fluid chamber 110 includes a fluid control valve 500 which is preferably a controllable valve (mechanic, electronic, etc.) It should be noted that controllable valves may be arranged to be in electrical communication with the processor 145 such that programmable software 149 can automatically control valve actuation in response to fluid 11 levels measured via the processor 145 and camera 140 of the fluid chamber 110.

Fluid control valve 500 is described in detail in U.S. Patent Application Publication No. 2022/0316605, filed Mar. 31, 2022 and published Oct. 6, 2022, which application is incorporated by reference herein in its entirety.

Generally, and in the representative embodiments, the aforementioned infusion device 10 could be included in an infusion delivery device 120. In some embodiments, the infusion delivery device 120 could comprise a fluid container 100 having a tube 102 extending from a distal end of the fluid container 100 and in fluid communication with the fluid container 100, a first check valve 125A arranged within the tube 102 and arranged downstream of the container, a pump 126, a pumping bulb 128, and a diaphragm 129, that is arranged to be manually stimulated to provide for pressure increases within the tube 102 in the downstream direction, a second check valve 125B in fluid communication with the pump 126 and arranged downstream of the pump 126 where the second check valve 125B allows for passive gravity flow of a fluid 11 therethrough in the downstream direction, a pressurizing device allowing pressure to be introduced to the pump 126 and a depressurizing device allowing pressure to be released from the pump 126, and optionally a fluid valve, where the second check valve 125B is arranged upstream from the fluid chamber 110 and is in fluid communication therewith.

The aforementioned infusion device 10 functions via three steps: 1. Pump priming; 2. Pumping; and 3. Gravity flow.

Pump priming generally requires that the pump 126 is physically collapsed, through a physical force, i.e., a force manually provided by a healthcare provider (e.g., by squeezing a flexible pressure bulb), thereby evacuating air from the downstream check valve, i.e., second check valve 125B.

Pumping occurs after the pump priming by first removing force from the pump 126 to allow the pump 126 to expand, thereby drawing fluid 11 into the pump 126 from the upstream check valve, i.e., first check valve 125A. Then the pump 126 is physically collapsed, thereby evacuating fluid 11 from the fluid and/or the downstream check valve 125B—repeated as many times as needed to achieve a desired flow rate of the infusion liquid (where the flow rate is calculated by the processor 145 of the infusion device 10).

After the pumping has stopped, and via the first and second check valves 125A, 125B, flow continues downstream via gravity, where the first and second check valves 125A, 125B can be adjusted to change the flow rate.

As such, it should be noted that the first and second check valves 125A, 125B of the infusion device 10 are adjustable to increase or decrease the fluid flow from the fluid container 100 to the fluid chamber 110.

In some embodiments of the present invention, the fluid chamber 110 (i.e., the drip chamber), is configured to be a flat-sided and rigid chamber-providing for optimal optical imaging clarity (of images and/or videos 146 captured by the camera 140). The fluid chamber 110 is arranged to be transparent and will include at least one planar (flat) wall 117 that is preferably arranged such that the camera 140 can capture images 146 of the fluid 11 therein and/or fluid drops 150 entering the fluid chamber 110 through the planar wall 117. The fluid chamber 110 includes the proximal end 111 and the distal end 119, where the fluid inlet port 122 is arranged within the proximal end (upstream end) 111 and the fluid outlet port 113 is arranged within the distal end (downstream end) 119. The fluid chamber 110 has a body 161 that defines the inner cavity 160 where fluid 11 is stored. The body 160 of the fluid chamber 110 is arranged to be transparent, preferably of substantially optically-clear construction and having flat and rigid walls 117 (i.e., not-flexible).

The present invention may also comprise a method of measuring volume of a fluid 11 entering a fluid chamber 110 of an infusion device 10 (“V₁”). Generally, in in the representative embodiments, the method is achieved by using image(s) 146 of drop(s) 150 (captured by the camera 140 and communicated to the processer 145) as the drops 150 fall through the air. The volume of the drop 150 is calculated based on the equation for an ellipsoid, using height and width derived from the drop image 146 aspect ratio and drop image 146 area. Absent any changes in delivery pressure, fluid 11 into the fluid chamber 110 will be equivalent to fluid 11 out of the fluid chamber 110, allowing for accurate flow measurement.

Generally, the method (V₁) involves:

Capturing one or more images 146 of a fluid drop 150 with a camera 140 after it separates from a drop former 123 (located within the fluid chamber's 110 cavity 160) and communicating the image 146 from the camera 140 to a processor 145. The processor 145, having software 148, 149 programed therein, measures the height (H) and width (W) of the fluid drop 150 in the respective image 146, allowing the software to then measure the area (A) of the fluid drop 150 in the respective image 146. The software 148, 149 uses the following equation to calculate the volume (V) of the fluid drop 150:

$$V = \frac{4}{3} \times A \times √ ( A \times \frac{W}{( π \times H )} )$$

The following method may be used in conjunction with the infusion device 10 recited in the appending claims and supra, assuming that the fluid chamber 110 includes a drop former 123 therein, the method comprising the steps of: (1) capturing at least one image 146 of a fluid drop 150 released from the drop former 123 via the camera 140; (2) communicating the at least one image 146 of the fluid drop 150 from the camera 140 to the processor 145; (3) measuring a height (H) and a width (W) of the at least one image 146 via the processor 145; (4) calculating an area (A) from the height (H) and the width (W) of the at least one image 146 via the processor 145; (5) calculating a volume (V) of the fluid drop 150 via the processor 145 using an equation of:

$$V = \frac{4}{3} \times A \times √ ( A \times \frac{W}{( π \times H )} ) ;$$

and, (6) summating calculated volumes of subsequent images 146 captured of additional fluid drops 150 via the processor 145 to determine a total fluid volume delivered to the fluid chamber 110.

The present invention may also comprise a method of combining the aforementioned method (V₁) with a method of determining fluid volume in the fluid chamber 110 (V₂), generally, the method (V₂) involves: (1) capturing an image 146 of a fluid 11 in the fluid chamber 110 via the camera 140; (2) communicating the image 146 from the camera 140 to the processor 145; and, (3) calculating a volume of the fluid 11 from the image 146 by: (4) measuring a height of the fluid 11; and, (5) multiplying the height by a cross-sectional area of the fluid chamber 110. It should be appreciated that the fluid chamber 110 will have a known cross-sectional area that dimension will be programmable to the processor 145 of the infusion device 10.

The present invention may further comprise a method of determining a volume of fluid 11 exiting the fluid chamber 110 (V₃), where the method (V₃) also employs the steps of both aforementioned methods (V₁ and V₂). Generally, the method (V₃) further comprises the steps of: a method of determining fluid volume in the fluid chamber 110 (V₂) comprising the steps of: capturing an image 146 of a fluid 11 in the fluid chamber 110 via the camera 140; communicating the image 146 from the camera 140 to the processor 145; and, calculating a volume of the fluid 11 from the image 146 by: measuring a height of the fluid 11; and, multiplying the height by a cross-sectional area of the fluid chamber 110; the method of determining a volume of fluid 11 exiting the fluid chamber 110 (V₃), comprising one of the steps of at least one of:
(1) subtracting an increase of the fluid volume in the fluid chamber 110 (V₂) from the fluid volume of the fluid 11 entering the fluid chamber 110 (V₁) via the processor 145; and,
(2) adding any decrease of the fluid volume in the fluid chamber 110 (V₂) from the fluid volume of the fluid 11 entering the fluid chamber 110 (V₁) via the processor 145.

In other words, software 148, 149 of the processor 145 determines the volume of fluid 11 in a fluid chamber 110 (i.e., V₂) based on: the fluid height in the fluid chamber 110, multiplied by the cross-sectional area of the fluid chamber 110.

In other words, the software 148, 149 of the processor 145 calculates fluid volume leaving (exiting) the fluid chamber 110 (i.e., V₃) based on: Subtracting any increase in fluid volume in the fluid chamber 110 from the volume entering the fluid chamber 110 over a given time period; and/or, adding any decrease in fluid volume in the fluid chamber 110 from the volume entering the fluid chamber 110 over a given time period.

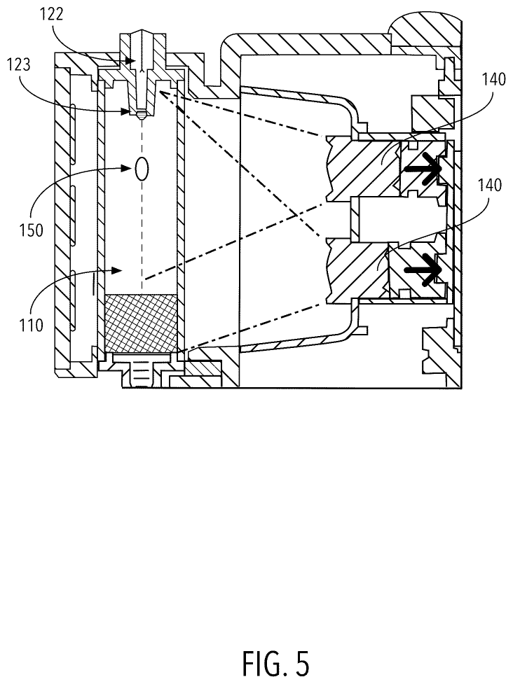

The present invention necessarily requires the camera 140 to have characteristics of a camera 140 suitable to record images of fluid drops with suitable resolution and clarity to perform as described in this disclosure. FIG. 5 illustrates a representative camera 140 system and that there may be more than one camera 140.

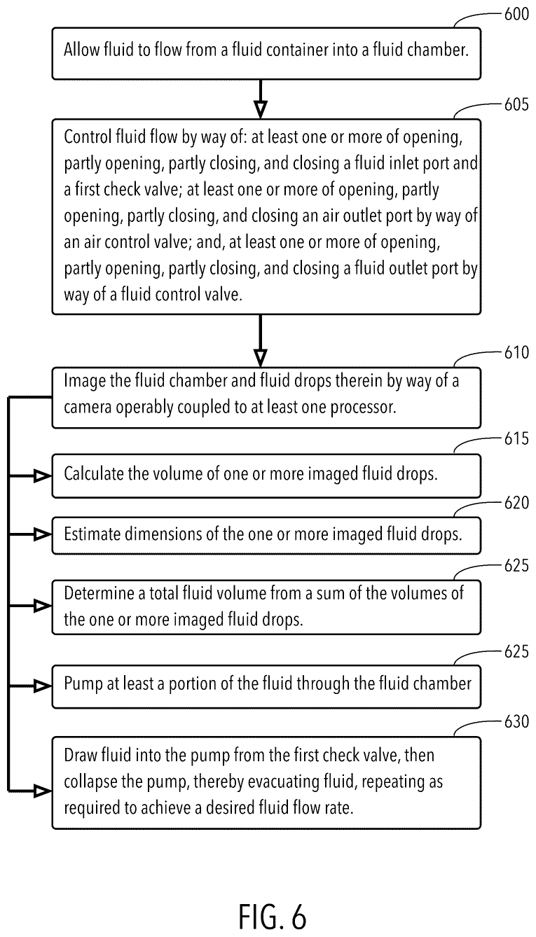

FIG. 6 illustrates a representative method of determining fluid volume flow through the infusion device 10 including the step of 600, allowing fluid to flow from the fluid container 100 into the fluid chamber 110. The method includes the step of 605 controlling fluid flow by way of: at least one or more of opening, partly opening, partly closing, and closing the fluid inlet port 122 and the first check valve 125A; at least one or more of opening, partly opening, partly closing, and closing the air outlet port 137 by way of the air control valve 135; and, at least one or more of opening, partly opening, partly closing, and closing the fluid outlet port 113 by way of the fluid control valve 500. The method includes the step of 610, imaging the fluid chamber 110 and fluid drops 150 therein by way of the camera 140 operably coupled to at least one processor 145. The method may include the step of 615, calculating the volume of one or more imaged fluid drops 150. The method may include the step of 620, estimating the dimensions of the one or more imaged fluid drops 150. The method may include the step of 625, determining a total fluid volume from a sum of the volumes of the one or more imaged fluid drops 150. The method may include the step of 630, pumping with the pump 126 at least a portion of the fluid through the fluid chamber 110. The method may include the step of 635, drawing fluid into the pump from the first check valve 125A, then collapsing the pump 126, thereby evacuating fluid, repeating as required to achieve a desired fluid flow rate.

It will be appreciated that various aspects of the invention and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

### COMPONENTS

10 Infusion Device
11 Fluid
12 Pump
25 External Container Portion of the Infusion Devise
100 Fluid Container
101 Proximal End of the Fluid Container
102 Tube
109 Distal End of the Fluid Container
110 Fluid Chamber
111 Proximal End of the Fluid Chamber
113 Fluid Outlet Port
114 Fluid Outlet Line
117 Planar (Flat) Wall
119 Distal End of the Fluid Chamber
120 Infusion Delivery Device
122 Fluid Inlet Port
123 Drop Former
125 Check Valve
125A First Check Valve
125B Second Check Valve
128 Pumping Bulb
129 Diaphragm
130 Air Pressure Release Device
135 Air Control Valve
137 Air Outlet Port
140 Camera
145 Processor
145A Primary Processor
145B Secondary Processor
146 Image
147 Venipuncture Cite
148 Calculating Software
149 Programmable Software
150 Fluid Drops
160 Internal Cavity
161 Body
166 Drip Chamber Cap
500 Fluid Control Valve
600-635 Representative method

What is claimed is:

1. An infusion device, comprising:
a fluid container having a proximal end and a distal end;
a fluid pump arranged downstream of said fluid container and in fluid communication with said fluid container;
a fluid chamber having a proximal end and a distal end, said fluid chamber arranged downstream of said fluid pump and in fluid communication with said fluid pump, said fluid chamber including:
a fluid inlet port arranged at said proximal end and in fluid communication with said fluid pump, said fluid inlet port having a first check valve arranged upstream;
an air outlet port arranged on a proximal end of said fluid chamber and in fluid communication with said distal end of said fluid container, said air outlet port having an air control valve arranged downstream in relation to said fluid container and further arranged upstream in relation to said fluid chamber; and,
a fluid outlet port arranged on a distal end of said fluid chamber and further including a fluid control valve, said fluid outlet port in fluid communication with a fluid outlet line arranged downstream of said fluid control valve; and,
a camera in electrical communication with at least one processor and having a field of view of said fluid chamber.

2. The fluid chamber recited in claim 1, wherein said fluid chamber is transparent, said fluid chamber comprising at least one planar wall.

3. The fluid chamber recited in claim 2, wherein said fluid chamber is rigid in construction.

4. The infusion device recited in claim 1, wherein said first check valve is arranged between said fluid chamber and said pump, a second check valve arranged between said pump and said fluid container, wherein said first check valve restricts fluid flow upstream and allows fluid flow downstream, wherein said second check valve restricts fluid flow upstream and allows fluid flow downstream.

5. The infusion device recited in claim 1, wherein said pump is a manual pump and allows downstream fluid flow therethrough, said pump further arranged with an air control valve.

6. The infusion device recited in claim 5, wherein said first check valve is arranged between said fluid chamber and said pump, a second check valve arranged between said pump and said fluid container, wherein said first check valve restricts fluid flow upstream and allows fluid flow downstream, wherein said second check valve restricts fluid flow upstream and allows fluid flow downstream.

7. The infusion device recited in claim 1, wherein said fluid chamber further comprises a drop former therein, said drop former in fluid communication and arranged downstream of said fluid inlet port.

8. The infusion device recited in claim 1, wherein said camera is adapted to image fluid drops falling within said fluid chamber.

9. The infusion device recited in claim 8, where said at least one processor and software disposed thereon is adapted to calculate the volume of one or more imaged fluid drops.

10. A method for measuring fluid volume of a fluid entering said fluid chamber (V₁) recited in claim 8, comprising the steps of:
capturing at least one image of a fluid drop released from said drop former via said camera;
communicating said at least one image of said fluid drop from said camera to said processor;
measuring a height (H) and a width (W) of said at least one image via said processor;
calculating an area (A) from said height (H) and said width (W) of said at least one image via said processor;
calculating a volume (V) of said fluid drop via said processor using an equation of:
$$V = \frac{4}{3} \times A \times √ ( A \times \frac{W}{( π \times H )} ) ;$$
summating calculated volumes of subsequent images captured of additional fluid drops via said processor to determine a total fluid volume delivered to said fluid chamber.

11. A method of determining fluid volume in said fluid chamber (V₂) recited in claim 1, comprising the steps of:
capturing an image of a fluid in said fluid chamber via said camera;
communication said image from said camera to said processor; and,
calculating a volume of said fluid from said image by:
measuring a height of said fluid; and,
multiplying said height by a cross-sectional area of said fluid chamber.

12. A method of determining a volume of fluid exiting said fluid chamber (V₃) recited in claim 10, further comprising:
a method of determining fluid volume in said fluid chamber (V₂) comprising the steps of:
capturing an image of a fluid in said fluid chamber via said camera;
communication said image from said camera to said processor; and,
calculating a volume of said fluid from said image by:
measuring a height of said fluid; and,
multiplying said height by a cross-sectional area of said fluid chamber;
said method of determining a volume of fluid exiting said fluid chamber (V₃), comprising one of the steps of at least one of:
subtracting an increase of said fluid volume in said fluid chamber (V₂) from said fluid volume of said fluid entering said fluid chamber (V₁) via said processor; and,
adding any decrease of said fluid volume in said fluid chamber (V₂) from said fluid volume of said fluid entering said fluid chamber (V₁) via said processor.

13. The infusion device recited in claim 5, wherein said manual pump includes a bulb.

14. The infusion device recited in claim 5, wherein said manual pump includes a diaphragm.

15. The infusion device recited in claim 5, wherein said manual pump is arranged with a passageway therein.

16. An infusion device, comprising:
a fluid container disposed above and in fluid communication with a fluid chamber;
the fluid chamber including:
a fluid inlet port and a first check valve adapted to control fluid flowing therethrough;
an air outlet port including an air control valve adapted to control airflow therethrough; and
a fluid outlet port and a fluid control valve adapted to control fluid outlet therethrough; and,
a camera operably coupled to at least one processor and adapted to image the fluid chamber and fluid drops therein.

17. The infusion device of claim 16, including as a part of the fluid chamber a pump.

18. The infusion device of claim 17, wherein the pump is adapted to be manually operated.

19. The infusion device of claim 17, wherein a second check valve is arranged between the pump and the fluid container.

20. The infusion device of claim 16, wherein the camera is adapted to image drops falling within the fluid chamber.

21. The infusion device of claim 16, wherein the at least one processor and software disposed thereon is adapted to calculate the volume of one or more imaged fluid drops.

22. The infusion device of claim 16 having a rigid fluid chamber.

23. The infusion device of claim 16, further including at least one valve adapted to release air.

24. The infusion device of claim 16, wherein the fluid chamber further includes a drop former therein beneath the fluid inlet port.

25. A method of determining fluid volume flow through an infusion device, comprising:
allowing fluid to flow from a fluid container into a fluid chamber;
controlling fluid flow by way of:
at least one or more of opening, partly opening, partly closing, and closing
a fluid inlet port and a first check valve;
at least one or more of opening, partly opening, partly closing, and closing
an air outlet port by way of an air control valve; and,
at least one or more of opening, partly opening, partly closing, and closing
a fluid outlet port by way of a fluid control valve; and,
imaging the fluid chamber and fluid drops therein by way of a camera operably coupled to at least one processor.

26. The method of determining fluid volume flow through an infusion device of claim 25, further including calculating the volume of one or more imaged fluid drops.

27. The method of determining fluid volume flow through an infusion device of claim 26, further including estimating the dimensions of the one or more imaged fluid drops.

28. The method of determining fluid volume flow through an infusion device of claim 26, further including determining a total fluid volume from a sum of the volumes of the one or more imaged fluid drops.

29. The method of determining fluid volume flow through an infusion device of claim 26, further including pumping with a pump at least a portion of the fluid through the fluid chamber.

30. The method of determining fluid volume flow through an infusion device of claim 29, further including drawing fluid into the pump from the first check valve, then collapsing the pump, thereby evacuating fluid, repeating as required to achieve a desired fluid flow rate.
